(19) United States
(12) Patent Application Publication
(10) Pub. No.: US 2021/0106279 A1
(43) Pub. Date: Apr. 15, 2021

(54) BODY MOVEMENT FEEDBACK SYSTEM AND METHOD

(71) Applicant: Upright Technologies Ltd., Yehud (IL)

(72) Inventors: Oded Cohen, Savyon (IL); Ori Fruhauf, Herzelia (IL); Liran Reller, Ganei Tikva (IL); Nir Porat, Jerusalem (IL); Lauren Finkelstein, Greenwich, CT (US)

(21) Appl. No.: 17/107,020

(22) Filed: Nov. 30, 2020

Related U.S. Application Data
(62) Division of application No. 15/749,335, filed on Jan. 31, 2018, now abandoned.
(60) Provisional application No. 62/201,914, filed on Aug. 6, 2015.

(51) Int. Cl. A61B 5/00; A61B 5/11; G06K 9/00; A61B 5/103

(52) U.S. Cl. CPC A61B 5/6832; G06F 3/011; A61B 5/1121; A61B 5/7425; A61B 5/6833; G06K 9/00342; A61B 5/103; A61B 5/6822; A61B 5/6823; A61B 5/6824; A61B 5/6828; A61B 5/0022; A61B 5/486; A61B 2562/166; A61B 2562/0219; A61B 2505/09; A61B 2562/164; A61B 5/6831

(57) ABSTRACT

A body movement feedback system including at least one position sensor operative to sense at least relative positions of mutually articulated body portions of a user in real time and providing sensor outputs, a mutually articulated body portion position processor, receiving the sensor outputs of the at least one position sensor and providing processor outputs representing relative movements of the mutually articulated body portions and a real time user feedback generator operative to generate predetermined feedback to the user in response to predetermined selected movements of the mutually articulated body portions.

### REFERENCE TO RELATED APPLICATIONS

Reference is hereby made to U.S. Provisional Patent Application Ser. No. 62/201,914, filed Aug. 6, 2015, entitled: BODY POSITION ASSESSMENT AND TRAINING DEVICE AND MANAGEMENT SYSTEM, the disclosure of which is hereby incorporated by reference and priority of which is hereby claimed pursuant to 37 CFR 1.78(a).

Reference is also hereby made to PCT Application No. PCT/IB2014/059041, filed Feb. 17, 2014, and entitled POSTURE DETECTION DEVICE, published Aug. 21, 2014 as PCT Published Patent Application WO 2014/125448 A1, the description of which is hereby incorporated by reference.

### FIELD OF THE INVENTION

The present invention relates to body motion training systems and methodologies.

### BACKGROUND OF THE INVENTION

There exist various systems and devices for body motion training.

### SUMMARY OF THE INVENTION

The present invention seeks to provide improved systems and devices for body motion training.

There is thus provided in accordance with a preferred embodiment of the present invention a body movement feedback system including at least one position sensor operative to sense at least relative positions of mutually articulated body portions of a user in real time and providing sensor outputs, a mutually articulated body portion position processor, receiving the sensor outputs of the at least one position sensor and providing processor outputs representing relative movements of the mutually articulated body portions and a real time user feedback generator operative to generate predetermined feedback to the user in response to predetermined selected movements of the mutually articulated body portions.

In accordance with a preferred embodiment of the present invention the body movement feedback system also includes a user feedback selector adapted to receive feedback selection inputs from a person and operative to assign selectable user feedbacks to selectable movements of the mutually articulated body portions.

Preferably, the at least one position sensor includes at least two body portion orientation sensors. Alternatively, the at least one position sensor includes at least one body portion orientation sensor and at least one bend sensor sensing bending between at least two mutually articulated body portions.

In accordance with a preferred embodiment of the present invention each of the at least two body portion orientation sensors measures the orientation of at least two body portions about an articulation joint joining the at least two body portions. Alternatively, each of the at least one body portion orientation sensor and at least one bend sensor measures the orientation of at least two body portions about an articulation joint joining the at least two body portions.

Preferably, the predetermined feedback includes at least one of tactile feedback, auditory feedback and visual feedback.

In accordance with a preferred embodiment of the present invention the at least one position sensor includes at least one first sensor mounted onto a user's skin at a first location and at least a second sensor mounted to the user's skin on a second location, the first and second locations being mutually separated and being on opposite sides of a vertex of a joint.

Preferably, the real time user feedback generator includes a feedback transducer operative to provide a real time notification to a user when at least one predetermined orientation of a joint is reached.

In accordance with a preferred embodiment of the present invention the body movement feedback system also includes a stretchable enclosure at least partially enclosing the at least one position sensor. Additionally, the stretchable enclosure also at least partially encloses the microprocessor and the feedback transducer.

In accordance with a preferred embodiment of the present invention the at least one position sensor is mounted onto the user's skin using at least one re-attachable fastener. Alternatively or additionally, the at least one position sensor is mounted onto the user's skin using at least one band.

Preferably, the body movement feedback system also includes a portable controller communicating wirelessly with the microprocessor. Additionally, the portable controller communicates wirelessly with a database.

In accordance with a preferred embodiment of the present invention the body movement feedback system also includes a substrate including at least first and second relatively non-elastic portions and at least one relatively elastic portion disposed therebetween and wherein the at least first and at least second sensors are mounted on the non-elastic portions of the substrate.

Preferably, the stretchable enclosure includes a substrate including at least first and second relatively non-elastic portions and at least one relatively elastic portion disposed therebetween and wherein at least first and at least second sensors are mounted on the non-elastic portions of the substrate.

There is also provided in accordance with another preferred embodiment of the present invention a method for body movement feedback including the steps of sensing at least relative positions of mutually articulated body portions of a user in real time and providing sensor outputs, receiving the sensor outputs and providing processor outputs representing relative movements of the mutually articulated body portions and generating predetermined feedbacks to the user in response to predetermined movements of the mutually articulated body portions.

In accordance with a preferred embodiment of the present invention the method for body movement feedback also includes receiving feedback selection inputs from a person and assigning selectable user feedbacks to selectable movements of the mutually articulated body portions.

Preferably, the sensing includes sensing the orientation of at least two body portions. Alternatively, the sensing includes sensing the orientation of at least one body portion and sensing bending between at least two mutually articulated body portions.

In accordance with a preferred embodiment of the present invention the sensing the orientation of at least two body portions includes measuring mutual orientation of at least two body portions about an articulation joint joining the at least two body portions. Alternatively, the sensing the orientation of at least one body portion and sensing bending between at least two mutually articulated body portions includes measuring mutual orientation of at least two body portions about an articulation joint joining the at least two body portions.

Preferably, the predetermined feedback includes at least one of tactile feedback, auditory feedback and visual feedback.

There is further provided in accordance with yet another preferred embodiment of the present invention a body position training device for measuring a user's joint orientation including at least one first sensor mounted onto a user's skin at a first location, at least a second sensor mounted to the user's skin on a second location, the first and second locations being mutually separated and being on opposite sides of a vertex of a joint, a microprocessor receiving inputs from the first and second sensors for calculating an orientation of the joint and a feedback transducer operative to provide a real time notification to a user when a predetermined orientation of the joint is reached.

Preferably, the first and second locations are each separated from the vertex by a distance sufficient to ensure that the first and second locations remain on opposite sides of the vertex at all orientations of the joint.

In accordance with a preferred embodiment of the present invention the body position training device for measuring a user's joint orientation also includes a stretchable enclosure at least partially enclosing the first and second sensors. Additionally, the stretchable enclosure also at least partially encloses the microprocessor and the feedback transducer.

Preferably, the at least first and at least second sensors are mounted onto the user's skin using at least one sticker. Alternatively, the at least first and at least second sensors are mounted onto the user's skin using at least one band.

In accordance with a preferred embodiment of the present invention the body position training device for measuring a user's joint orientation also includes a portable controller communicating wirelessly with the microprocessor.

In accordance with a preferred embodiment of the present invention the body position training device for measuring a user's joint orientation also includes a substrate including at least first and second relatively non-elastic portions and at least one relatively elastic portion disposed therebetween and wherein the at least first and at least second sensors are mounted on the non-elastic portions of the substrate. Additionally or alternatively, the stretchable enclosure includes a substrate including at least first and second relatively non-elastic portions and at least one relatively elastic portion disposed therebetween and wherein the at least first and at least second sensors are mounted on the non-elastic portions of the substrate.

### BRIEF DESCRIPTION OF PREFERRED EMBODIMENTS

The present invention will be understood and appreciated more fully from the following detailed description, taken in conjunction with the drawings in which:

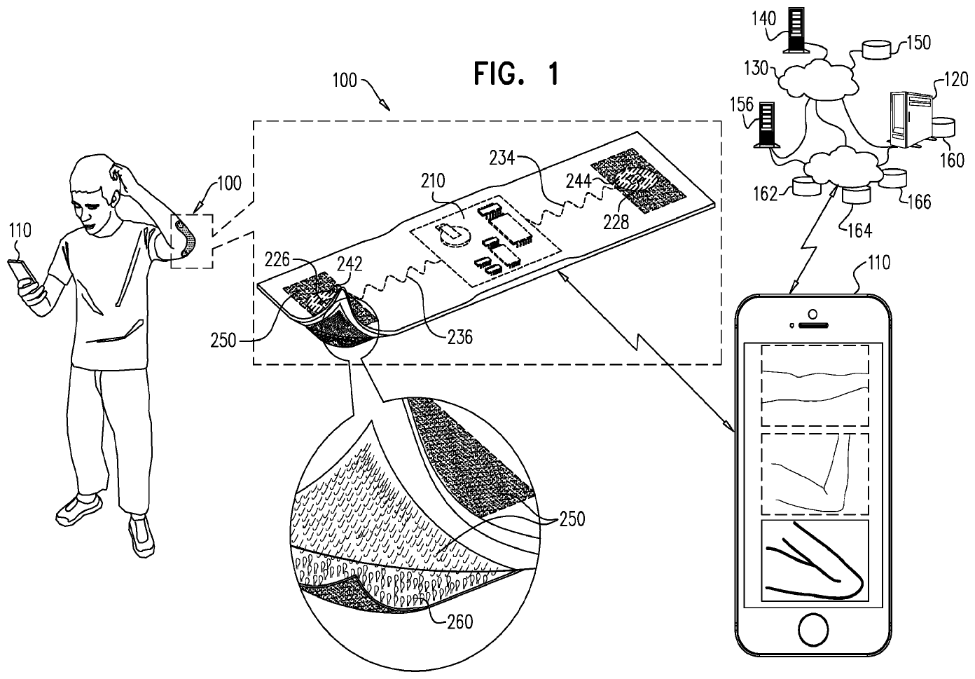

FIG. 1 is a simplified illustration of a body position training system including a body-mounted body position training device, a portable control device and a management interface workstation;

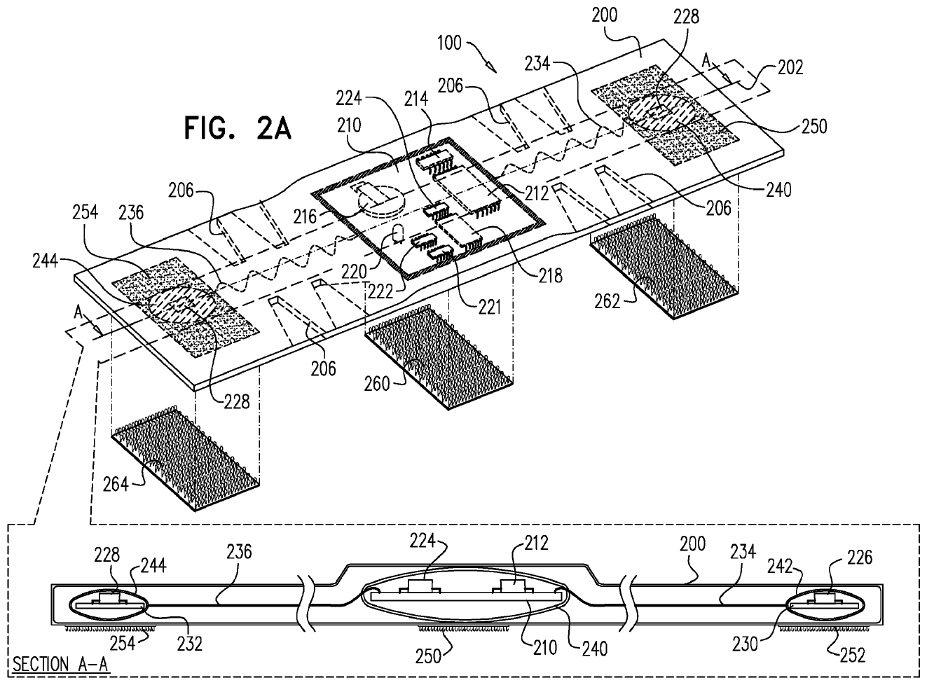

FIGS. 2A and 2B are simplified respective top view and bottom view perspective illustrations of a body position training device constructed and operative in accordance with a preferred embodiment of the present invention;

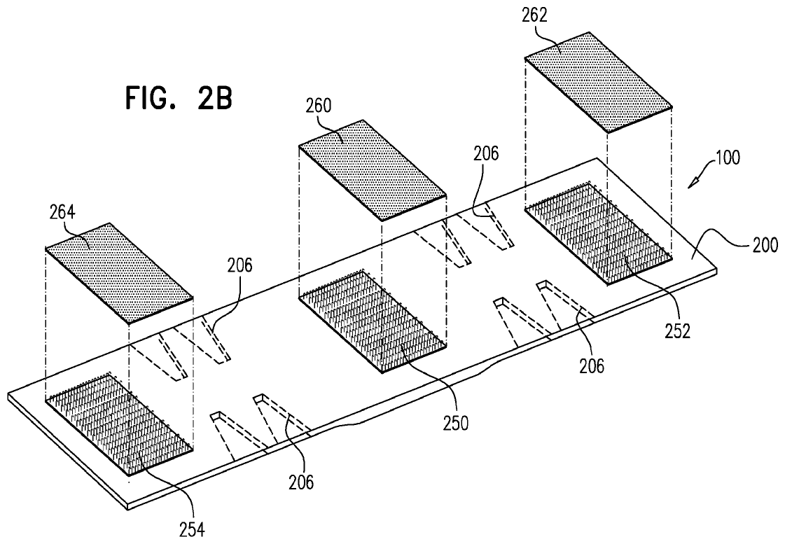

FIGS. 3A and 3B are simplified respective top view and bottom view perspective illustrations of a body position training device constructed and operative in accordance with another preferred embodiment of the present invention;

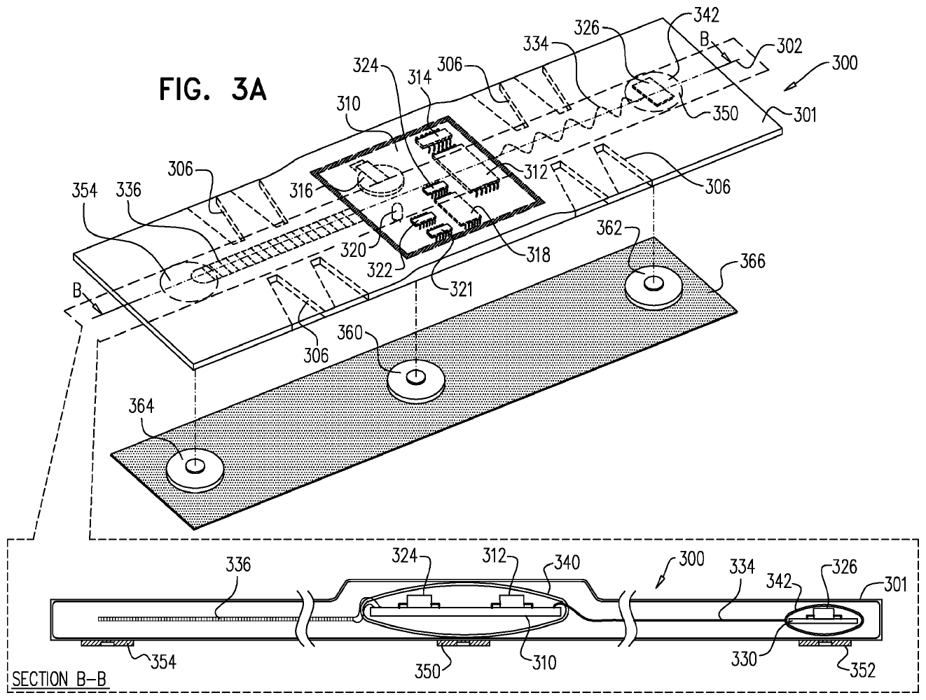

FIGS. 4A and 4B are simplified respective top view and bottom view perspective illustrations of a body position training device constructed and operative in accordance with yet another preferred embodiment of the present invention;

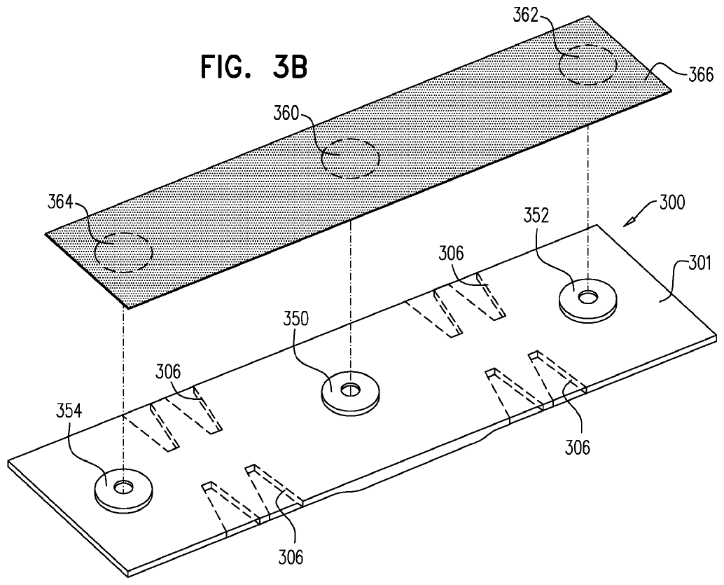

FIGS. 5A and 5B are simplified pictorial illustrations of two phases in the operation of the body position training system for training;

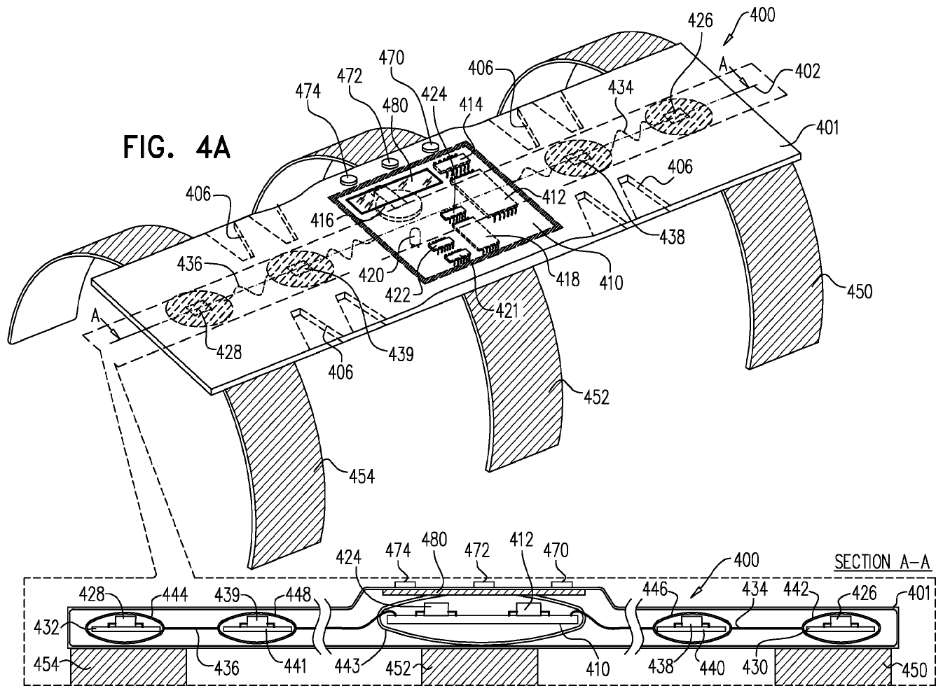

FIGS. 6A and 6B are simplified pictorial illustrations of two phases in the operation of the body position training system for training;

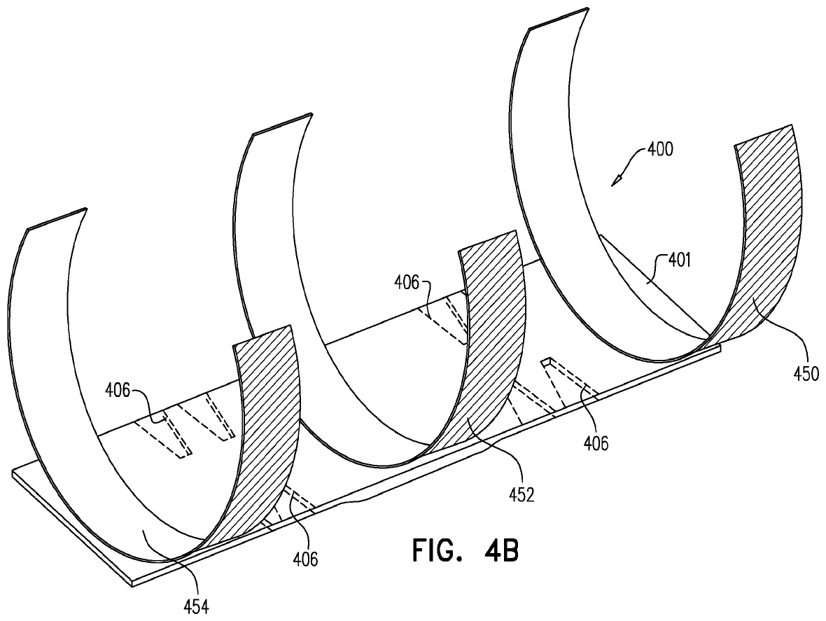

FIGS. 7A and 7B are simplified front and back body outline illustrations showing examples of body locations for possible mounting and operation of the body-mounted body position training device of FIGS. 1-6B;

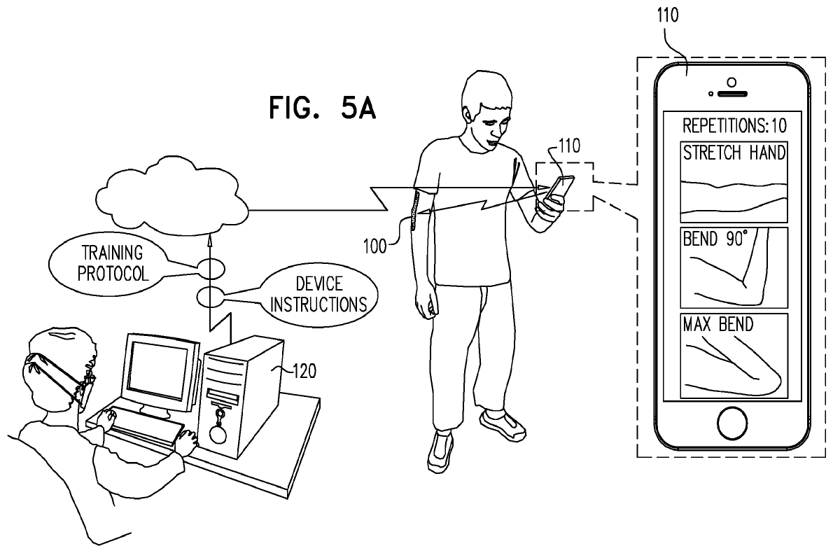

FIGS. 8A and 8B are simplified illustrations of two body positions which may be sensed by an elbow-mounted body position training device of FIGS. 1-5B;

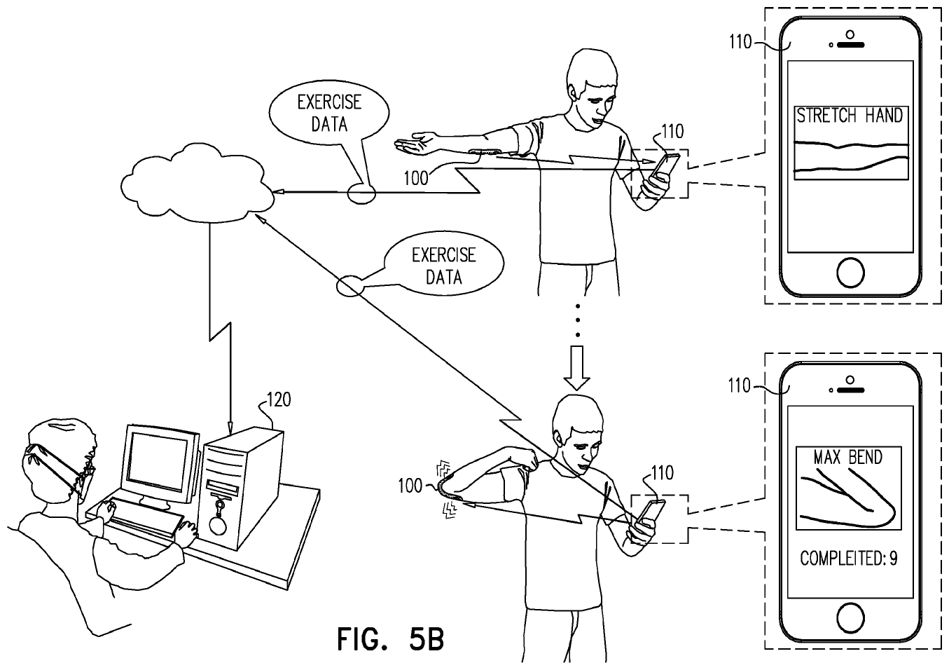

FIGS. 9A and 9B are simplified illustrations of two body positions which may be sensed by an ankle-mounted body position training device of FIGS. 1-5B;

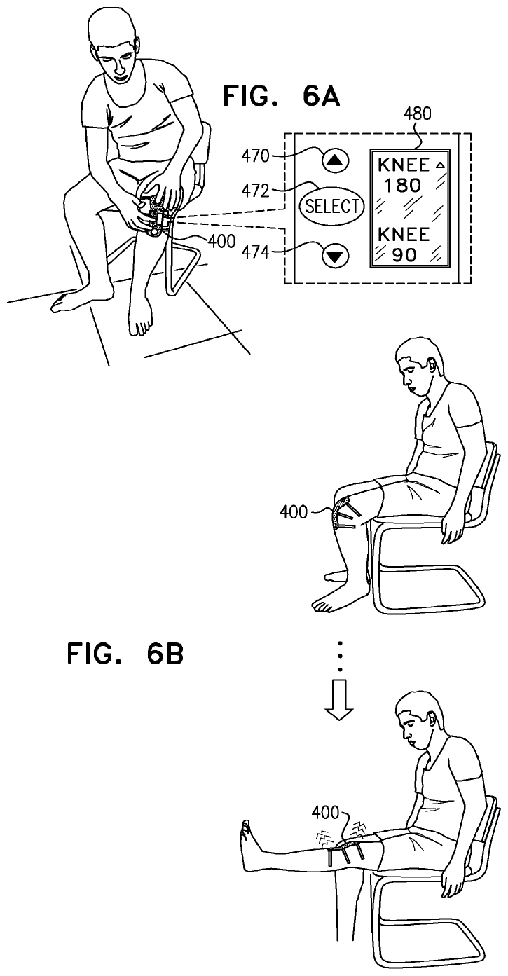

FIGS. 10A and 10B are simplified illustrations of two body positions which may be sensed by a knee-mounted body position training device of FIGS. 1-5B;

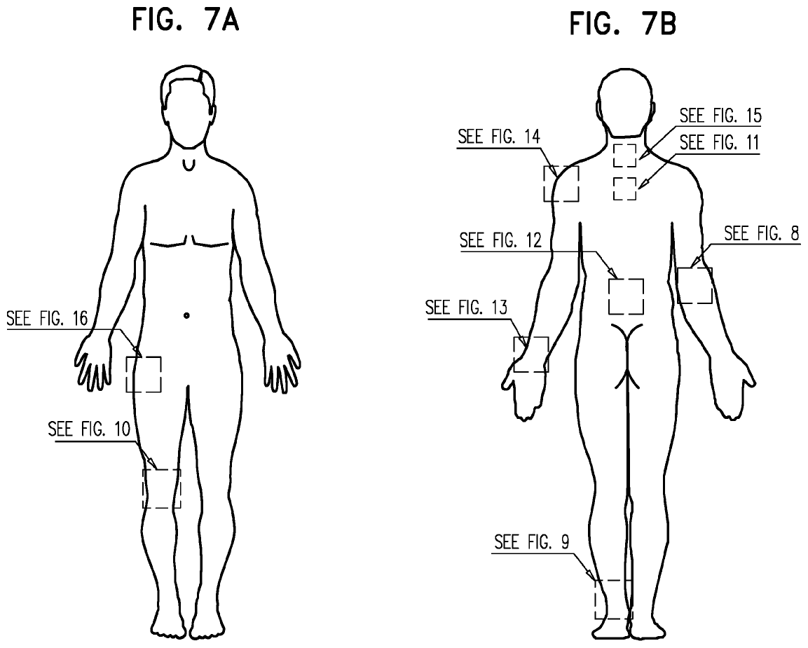

FIGS. 11A and 11B are simplified illustrations of two body positions which may be sensed by an upper back-mounted body position training device of FIGS. 1-5B;

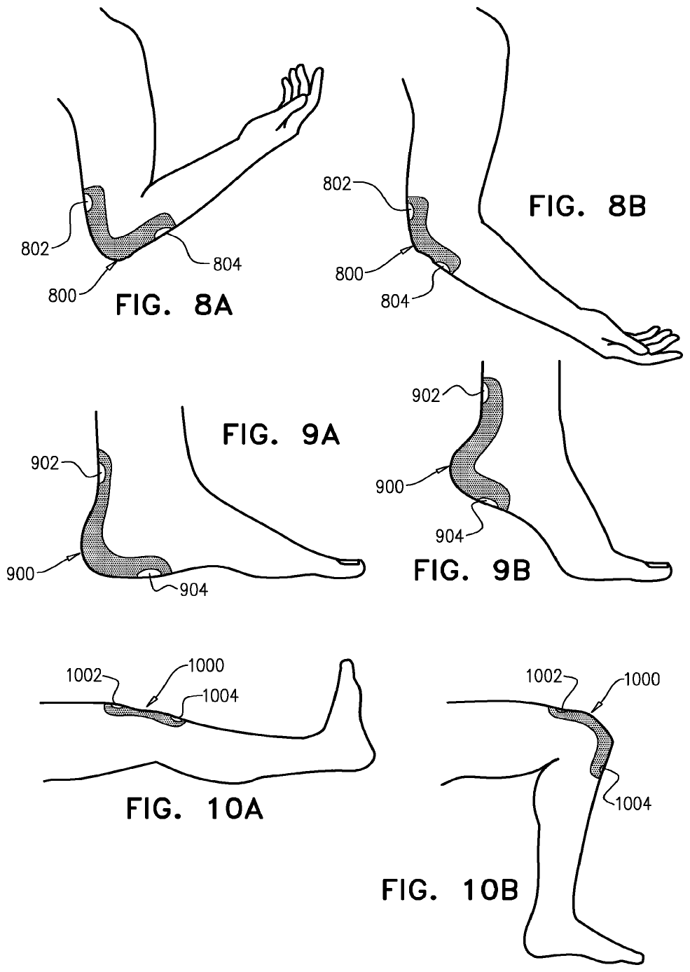

FIGS. 12A and 12B are simplified illustrations of two body positions which may be sensed by a lower back-mounted body position training device of FIGS. 1-5B;

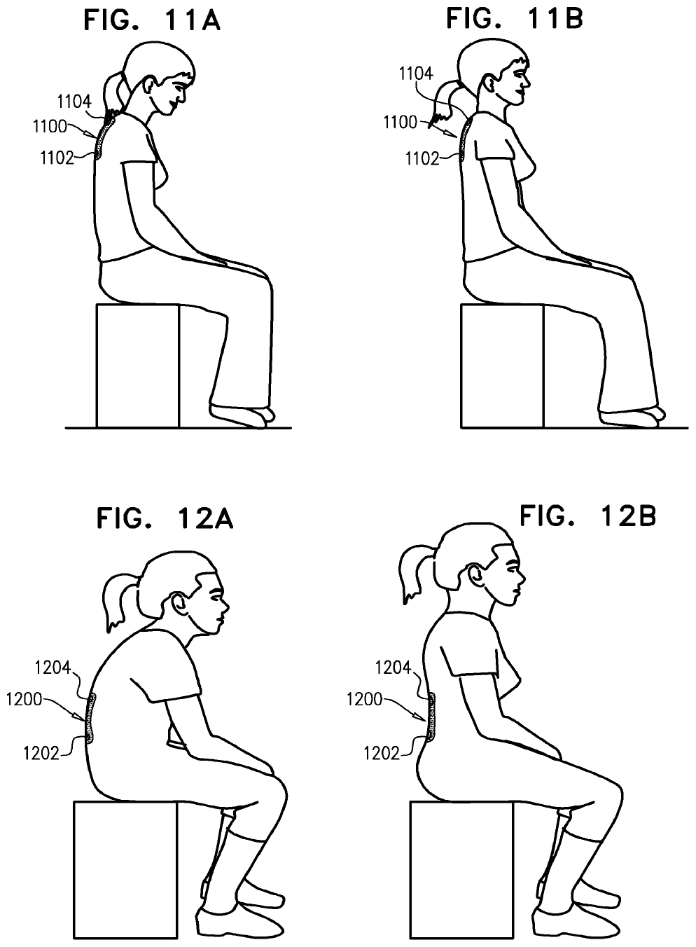

FIGS. 13A, 13B and 13C are simplified illustrations of three body positions which may be sensed by a wrist-mounted body position training device of FIGS. 1-5B;

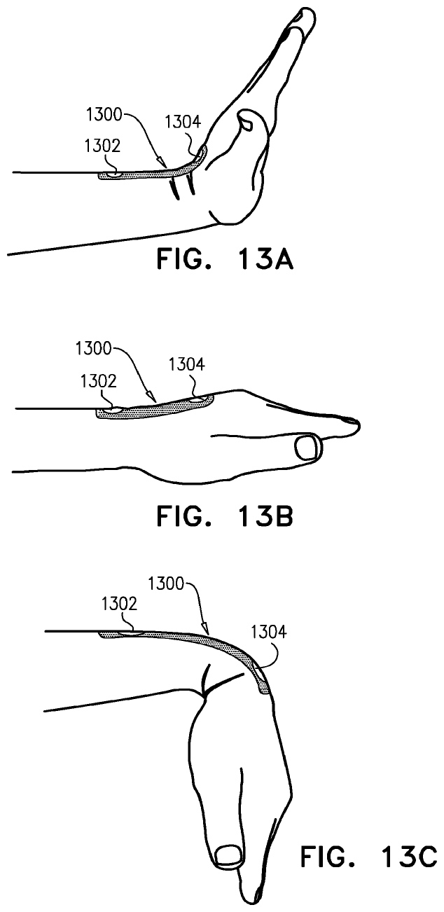

FIGS. 14A and 14B are simplified illustrations of two body positions which may be sensed by a shoulder-mounted body position training device of FIGS. 1-5B;

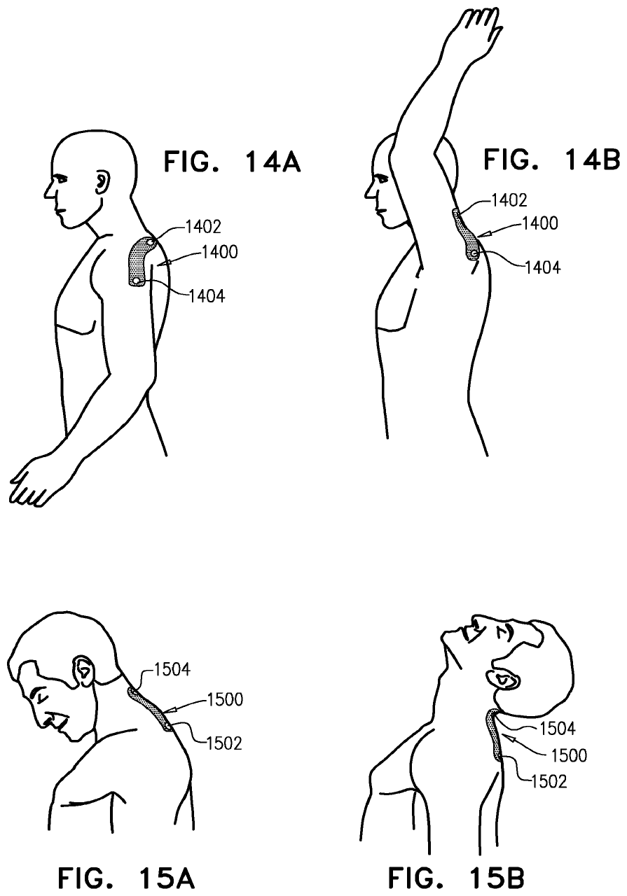

FIGS. 15A and 15B are simplified illustrations of two body positions which may be sensed by a neck-mounted body position training device of FIGS. 1-5B; and

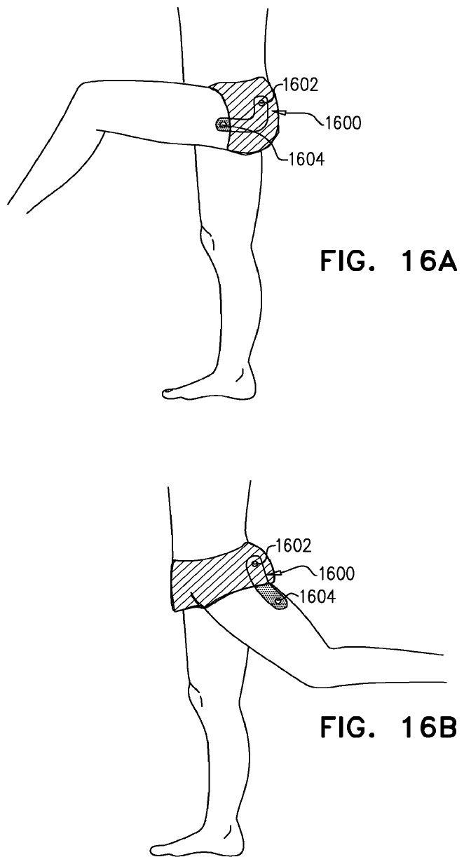

FIGS. 16A and 16B are simplified illustrations of two body positions which may be sensed by a hip-mounted body position training device of FIGS. 1-5B.

### DETAILED DESCRIPTION OF PREFERRED EMBODIMENTS

Reference is now made to FIG. 1, which is a simplified illustration of a body position training system including a body-mounted body position training device 100 that is preferably worn by the user, a portable control device 110 that is preferably controlled by the user and a management interface workstation 120 that is preferably controlled by a practitioner such as a physician, physical therapist or sports coach.

The body-mounted body position training device 100 is preferably controlled by portable control device 110, which may be embodied in a generic smartphone, a smartwatch or any other suitable mobile communication device, which may or may not be specifically designed for use in the system.

The portable control device 110 preferably communicates wirelessly with the body-mounted body position training device 100 and wirelessly, typically via the Internet, with the management interface workstation 120. The user preferably is the only person who can control the body position training device 100. Alternatively or additionally, a practitioner may control the body position training device 100 using the management interface workstation 120 and the portable control device 110 or directly via the portable control device 110.

The management interface workstation 120 preferably interfaces with one or more other healthcare provider networks 130. A practitioner can access the management interface workstation 120 directly using suitable software and/or from a general healthcare platform 140, typically via a healthcare provider network 130. Various health management databases 150 are connected via networks 130, which may be connected to one or more cloud servers 156.

The management interface workstation 120 may provide selectable access to one or more of the following: a practitioner database 160, a user database 162, a training program database 164 and an exercise protocol database 166. Via the management interface workstation 120, a practitioner can send training programs from a training program database 164 and exercise protocols from database an exercise protocol data base 166 to be carried out by the user as well as other data and information to the portable control device 110.

From the portable control device 110, programmed commands and conditions related to the assigned training programs and exercise protocols can be sent to the body position training device 100. According to these commands and conditions, the body position training device 100 collects data from the user and administers feedback to the user during training and exercise. For example, such data and feedback could be related to the accuracy of the user's performance of an assigned exercise.

Information sent to a user's portable control device 110, such as exercise protocols, training programs and data collected by the body position training device 100, is preferably stored in the user's profile in the user database 162. Furthermore, the aforementioned user profile can preferably be accessed by the practitioner via the interface workstation 120.

During use of the system, data collected by the body position training device 100 is sent wirelessly by the body position training device 100 to the portable control device 110. From the portable control device 110, information and data preferably is exported to the management interface workstation 120. From the management interface workstation 120, information can also be sent to healthcare provider networks 130 to be stored in health management databases 150.

An example of how a practitioner, in this case a physiotherapist, may instruct a user, in this case a client, to use the body position training device 100 is as follows: The practitioner assigns an exercise protocol of elbow stretching and bending exercises and a training program of performing two sets of specific elbow exercises per day including 10 repetitions per set. The practitioner instructs the user to wear the body position training device 100 on his elbow while performing the exercises in order to ensure that the exercises are performed correctly.

Preferably, the body position training device 100 provides feedback to the user. The body position training device 100 preferably transmits data acquired during the exercises to the portable control device 110 for viewing by the user and, preferably via the portable control device 110 to the management interface workstation 120 for consideration by the practitioner and archiving in one or more databases, such as health management databases 150.

Reference is now made additionally to FIGS. 2A and 2B, which illustrate, in greater detail, the embodiment of the body position training device 100 shown in FIG. 1.

As seen in FIGS. 1-2B, the body position training device 100 preferably includes an enclosure 200, arranged along a longitudinal axis 202, which is preferably bendable through at least 90 degrees about an axis perpendicular to the longitudinal axis 202 and stretchable by at least 10% and preferably by 25% along longitudinal axis 202. A preferred material for enclosure 200 is Neoprene Foam manufactured by Marco International Company, Marco Product Inc from 78 Bunsen, Irvine, Calif. 92618. Preferably the length of the enclosure 200 is preferably between 6-14 cm and more preferably between 8-12 cm and the width of the enclosure 200 is preferably between 1-4 cm and more preferably between 2-3 cm, the thickness of the enclosure material is up to 1 mm. Stretchability may be enhanced by providing cut outs 206, shown in dashed lines.

Disposed within enclosure 200 is a printed circuit board 210, preferably of dimensions 25 mm×25 mm, on which are preferably mounted a microprocessor 212 as well as a tactile feedback generator 214, a battery 216, a wireless transceiver 218, a visual feedback generator 220, a memory 221 and an auditory feedback generator 222, all coupled to microprocessor 212. Optionally an orientation sensor 224 may also be mounted on printed circuit board 210 and coupled to the microprocessor 212.

Also disposed within enclosure 200 adjacent ends thereof are a pair of orientation sensors, here designated by reference numerals 226 and 228. Orientation sensors 226 and 228 are not mounted on printed circuit board 210 and are not rigidly coupled to printed circuit board 210. Orientation sensors 226 and 228 are preferably each mounted on a printed circuit board, here respective designated by reference numerals 230 and 232. Orientation sensors 226 and 228 are preferably electrically connected to microprocessor 212 via respective flexible conductors 234 and 236. Orientation sensors 224, 226 and 228 are preferably model numbers BHI 160 or BMI 160 manufactured by Bosch Sensor Tech from Gerhard-Kindler-StraBe 9 72770 Reutlingen/Kusterdingen, Germany.

Each of printed circuit boards 210, 230 and 232 is preferably enclosed in respective relatively rigid protective capsules 240, 242 and 244, which preferably provide hermetic sealing and mechanical protection to the orientation sensors and other components mounted thereon.

Disposed on an underside outside surface of enclosure 200 underlying each of capsules 240, 242 and 244 are fastener strips 250, 252 and 254 respectively. Fastener strips 250, 254 and 256 are preferably VELCRO® hook strips having an adhesive backing, which adheres to the underside outside surface of enclosure 200. Fastener strips 250, 252 and 254 are configured to be removably attachable to corresponding fastener strips 260, 262 and 264, preferably VELCRO® loop strips, which are adhesively attachable to the skin of a user. Fastener strips 260, 262 and 264 are preferably disposable and are not reused. The adhesive backing is preferably, for example, are #2475 [formerly MSX-6936A] Single Coated Medical Silicone Adhesive Tape on Liner, commercially available from the 3M Company of Saint Paul, Minn., United States.

Orientation sensors 224, 226 and 228 preferably include an accelerometer, for measuring the angle relative to gravity, and/or a gyroscope, for measuring the orientation of the body position training device 100.

It is appreciated that printed circuit board 210 may be obviated and that microprocessor 212, as well as a tactile feedback generator 214, battery 216, wireless transceiver 218, visual feedback generator 220, memory 221 and auditory feedback generator 222 may be mounted on printed circuit board 230 or 232.

Reference is now made additionally to FIGS. 3A and 3B, which illustrate an alternative embodiment of the body position training device 100 shown in FIG. 1.

As seen in FIGS. 3A & 3B, the body position training device, here designated by reference numeral 300, preferably includes an enclosure 301, arranged along a longitudinal axis 302, which is preferably bendable through at least 100 degrees about an axis perpendicular to the longitudinal axis 302 and stretchable by at least 10% and preferably by 25% along longitudinal axis 302. A preferred material for enclosure 301 is Neoprene Foam manufactured by Marco International Company, Marco Product Inc from 78 Bunsen, Irvine, Calif. 92618. Preferably the length of the enclosure 301 is between 6-14 cm and more preferably between 8-12 cm, the width of the enclosure 200 is preferably between 1-4 cm and more preferably between 2-3 cm and the thickness of the enclosure material is up to 1 mm. Stretchability may be enhanced by providing cutouts as shown at 306 in FIGS. 3A and 3B.

Disposed within enclosure 301 is a printed circuit board 310, preferably of dimensions 25 mm×25 mm, on which are preferably mounted a microprocessor 312 as well as a tactile feedback generator 314, a battery 316, a wireless transceiver 318, a visual feedback generator 320, a memory 321 and an auditory feedback generator 322, all coupled to microprocessor 312. Optionally an orientation sensor 324 may also be mounted on printed circuit board 310 and coupled to the microprocessor 312.

Also disposed within enclosure 301, adjacent an end thereof, is an orientation sensor, here designated by reference numerals 326. Orientation sensor 326 is not mounted on printed circuit board 310 and is not rigidly coupled to printed circuit board 310. Orientation sensor 326 is preferably mounted on a printed circuit board 330. Orientation sensor 326 is preferably electrically connected to microprocessor 312 via a flexible conductor 334. Orientation sensors 324 and 326 are preferably BHI 160 or BMI 160 manufactured by Bosch Sensor Tech from Gerhard-Kindler-Straße 9 72770 Reutlingen/Kusterdingen, Germany.

Additionally disposed within enclosure 301 is a bend sensor 336, preferably Tactilus® Flex manufactured by Sensor Products Inc from 300 Madison Avenue Madison, N.J. 07940 USA, which is preferably fixedly mounted at one end thereof to printed circuit board 310.

Each of printed circuit boards 310 and 330 is preferably enclosed in respective relatively rigid protective capsules 340 and 342, which preferably provide hermetic sealing and mechanical protection to the orientation sensors and other components mounted thereon.

Disposed on an underside outside surface of enclosure 301 underlying each of capsules 340 and 342 and underlying an end of enclosure 301 opposite to the end at which capsule 350 is located are snap fastener elements 350, 352 and 354 respectively. Snap fastener elements 350, 352 and 354 are configured to be removably attachable to corresponding snap fastener elements 360, 362 and 364, which are fixedly mounted onto a flexible, stretchable mounting strip 366, such as inelastic cotton manufactured by STRENGTHTAPE® from 6753 Engle Road Middleburg Heights, Ohio 44130 United States of America. Stretchable mounting strip 366 and the snap fastener elements 360, 362 and 364 are preferably disposable and not reused. It is understood that other types of fasteners may be used, such as metal sheets, magnets, textile and buttons, which are adhesively attachable to the skin of a user. A suitable adhesive material is, for example, are #2475 [formerly MSX-6936A] Single Coated Medical Silicone Adhesive Tape on Liner, commercially available from the 3M Company, of Saint Paul, Minn., United States.

Orientation sensors 324 and 326 may include an accelerometer, for measuring the angle relative to gravity, and/or a gyroscope, for measuring the orientation of the body position training device 300.

Bend sensor 336 for measuring the degree of curvature of the body position training device 300 may include a force sensor such as FlexiForce A401 Sensor, manufactured by Tekscan, Inc. from, 307 West First Street South Boston, Mass. 02127-1309, USA, a flex sensor such as flex sensor manufactured by Spectra Symbol from 3101 W. 2100 S. Salt Lake City, Utah 84119 USA, used to measure the flexure of the body-mounted body position training device 100; a stretch sensor such as Leap stretch sensor from Science and Technology Park, Diplomvej 381, 2800 Kgs. Lyngby, Denmark, used to measure the stretch of a component of the body-mounted body position training device 100 and a strain gauge such as Zhonghang Electronic Measuring Instruments Co., LTD from, No. 166, West Avenue, Chang'an District, Xi'an, Shaanxi, China, used to measure the strain on the body-mounted body position training device 100.

It is appreciated that printed circuit board 310 may be obviated and microprocessor 312, as well as a tactile feedback generator 314, battery 316, wireless transceiver 318, visual feedback generator 320, memory 321 and auditory feedback generator 322 may be mounted on printed circuit board 330.

Reference is now made to FIGS. 4A and 4B, which are simplified respective top view and bottom view perspective illustrations of a body position training device constructed and operative in accordance with yet another preferred embodiment of the present invention.

As seen in FIGS. 4A and 4B, the body position training device 400 preferably includes an enclosure 401, arranged along a longitudinal axis 402, which is preferably bendable through at least 100 degrees about an axis perpendicular to the longitudinal axis 402 and stretchable by at least 10% and preferably by 25% along longitudinal axis 402. A preferred material for enclosure 401 is Neoprene Foam manufactured by Marco International Company, Marco Product Inc from 78 Bunsen, Irvine, Calif. 92618. Preferably the length of the enclosure 401 is preferably between 6-14 cm and more preferably between 8-12 cm, the width of the enclosure 401 is preferably between 1-4 cm and more preferably between 2-3 cm and the thickness of the enclosure material is up to 1 mm. Stretchability of the enclosure 401 may be enhanced by providing cut outs 406, shown in dashed lines.

Disposed within enclosure 401 is a printed circuit board 410, preferably of dimensions 25 mm×25 mm, on which are preferably mounted a microprocessor 412 as well as a tactile feedback generator 414, a battery 416, optionally a wireless transceiver 418, a visual feedback generator 420 and an auditory feedback generator 422, all coupled to microprocessor 412. Optionally an orientation sensor 424 may also be mounted on printed circuit board 410 and coupled to the microprocessor 412.

Also disposed within enclosure 401 adjacent ends thereof are a pair of orientation sensors, here designated by reference numerals 426 and 428. Orientation sensors 426 and 428 are not mounted on printed circuit board 410 and are not rigidly coupled to printed circuit board 410. Orientation sensors 426 and 428 are preferably each mounted on a printed circuit board, here respective designated by reference numerals 430 and 432. Orientation sensors 426 and 428 are preferably electrically connected to microprocessor 412 via respective flexible conductors 434 and 436.

Also disposed within enclosure 401 are additional intermediate orientation sensors, here designated by reference numerals 438 and 439. Orientation sensors 438 and 439 are not mounted on printed circuit board 410 and are not rigidly coupled to printed circuit board 410. Orientation sensors 438 and 439 are preferably each mounted on a printed circuit board, here respective designated by reference numerals 440 and 441. Orientation sensors 438 and 439 are preferably electrically connected to microprocessor 412 via respective flexible conductors 434 and 436.

Orientation sensors 424, 426, 428, 438 and 439 are preferably either of model numbers BHI 160 or BMI 160 manufactured by Bosch Sensor Tech from Gerhard-Kindler-Straße 9 72770 Reutlingen/Kusterdingen, Germany.

Each of printed circuit hoards 410, 430 and 432, 440 and 441 is preferably enclosed in a respective relatively rigid protective capsule 443, 442, 444, 446 and 448, which preferably provide hermetic sealing and mechanical protection to the orientation sensors and other components mounted thereon.

Disposed on an underside outside surface of enclosure 401 underlying each of capsules 442, 443 and 444 are fastener strips 450, 452 and 454 respectively. Fastener strips 450, 452 and 454 are preferably of a type usable for wrist or ankle mountable spring-like bracelets or equivalents.

Orientation sensors 424, 426, 428, 438 and 439 preferably include an accelerometer, for measuring angular orientation relative to gravitational acceleration, and/or a gyroscope, for measuring the orientation of the body position training device 400.

Also disposed within enclosure 401 are buttons 470, 472 and 474 and display 480.

Body position training device 400 preferably is switched on and off by a long press on button 472. Buttons 470 and 474 are preferably employed to move a menu displayed on display 480 up and down. Button 472 may be used also for menu selection. In contrast to body position training device 100 and 300 shown in FIGS. 1A-3B, body position training device 400 is preferably operated without a portable control device 110.

It is appreciated that printed circuit board 410 may be obviated and microprocessor 412, as well as a tactile feedback generator 414, battery 416, wireless transceiver 418, visual feedback generator 420, memory 421, auditory feedback generator 422, buttons 470, 472 and 474 and display 480 may be mounted on printed circuit board 430, 432, 440 and/or 441.

Reference is now made to FIGS. 5A and 5B, which are simplified pictorial illustrations of two phases in the operation of the body position training system for training. For simplicity, the description refers to body position training device 100 shown in FIG. 1-2B. It is appreciated that the description which follows is also applicable to the operation of body position training device 300 shown in FIGS. 3A and 3B.

FIG. 5A illustrates an initial phase in which training instructions are downloaded to the portable control device 110. The download may be from a management interface workstation 120 on the basis of instructions entered by a practitioner. Alternatively, the download may be from a website or any other suitable source of training instructions.

The training instructions downloaded to the portable control device 110 preferably include instructions to the user, which are intended for display on the portable control device 110, as shown as well as instructions to the body position training device, such as body-mounted body position training device 100, which are preferably relayed to the body-mounted body position training device 100 via the portable control device 110. The instructions to the body position training device preferably include at least one of:
1. Feedback generation instructions based on a predetermined operative orientation of the body-mounted body position training device 100, such as a 90 degree bend of the elbow.
2. Feedback generation instructions based on exceedance of a predetermined operative orientation threshold of the body-mounted body position training device 100, such as a bend of the elbow exceeding 90 degrees.
3. Feedback generation instructions based on a reaching a plurality of predetermined operative orientations of the body-mounted body position training device 100, such as full extension of the arm, 90 degree orientation at the elbow and maximum arm bend.
4. Feedback generation instructions based on a completion of a predetermined number of predetermined movements of the body-mounted body position training device 100, such as completion of each of a plurality of arm bends through a predetermined angular range.

FIG. 5B illustrates a subsequent phase in which an exercise, instructions regarding which were earlier downloaded to the portable control device 110 and to the body-mounted body position training device 100, is being carried out. FIG. 5B shows an example corresponding to example 4 above wherein feedback is generated in response to completion of a predetermined number of predetermined movements of the body-mounted body position training device 100.

As seen in FIG. 5B, the user initially extends his arm as instructed by the image displayed on the portable control device 110 and finally fully bends his elbow as further instructed by the image displayed on the portable control device 110, thus completing the exercise downloaded as shown in FIG. 5A. In accordance with a preferred embodiment of the present invention the user receives feedback in real time upon completion of the exercise. Additionally, preferably, a report confirming completion of the exercise is sent from the body-mounted body position training device 100 via the portable control device to the management interface workstation 120 and to the practitioner.

Reference is now made to FIGS. 6A and 6B, which are simplified pictorial illustrations of two phases in the operation of the body position training system shown in FIG. 4A-4B for training.

FIG. 6A illustrates an initial phase in which training instructions are stored within memory 421. Optionally, training instructions may be downloaded via wireless transceiver 418 to the body position training device 400. The download may be from a portable control device (not shown).

The training instructions are intended for display on the display 480 of the body position training device 400, as shown in FIG. 6A as well as instructions to the body position training device 400. The instructions to the body position training device preferably include at least one of:
1. Feedback generation instructions based on a predetermined operative orientation of the body-mounted body position training device 400, such as upon straightening of the knee.
2. Feedback generation instructions based on exceedance of a predetermined operative orientation threshold of the body-mounted body position training device 400, such as at half the way between straightening of the knee and 90 degrees knee bend.
3. Feedback generation instructions based on a reaching a plurality of predetermined operative orientations of the body-mounted body position training device 400, such as at a 90 degree knee bend and at a full straightening of the knee.
4. Feedback generation instructions based on a completion of a predetermined number of predetermined movements of the body-mounted body position training device 400, such as completion of each of a plurality of knee bends through a predetermined angular range.

FIG. 6B illustrates a subsequent phase in which an exercise, instructions regarding which were earlier stored within memory 480 of body-mounted body position training device 400, is being carried out. FIG. 5B shows an example corresponding to example 1 above wherein feedback is generated in response to reaching 180 degrees of knee straightening.

As seen in FIG. 6B, the user initially bends his knee at 90 degrees as instructed by the prompt displayed on the body position training device 400 and finally fully straightening his knee as further instructed by a prompt displayed on display 480 of the body position training device 400 thus completing a repetition of an exercise downloaded as shown in FIG. 6A. In accordance with a preferred embodiment of the present invention the user receives feedback notification in real time upon straightening his knee at 180 degrees. Additionally and optionally, a report confirming completion of the exercise is sent from the body-mounted body position training device 400 to portable control device.

Reference is now made to FIGS. 7A and 7B, which are simplified front and back body outline illustrations showing examples of body joint locations for possible mounting and operation of the body-mounted body position training device of FIGS. 1-6B.

As seen in FIGS. 8A and 8B, for example, a body mounting body position training device of any of the types described above with reference to FIGS. 1-6B, may be mounted on a user's elbow, as described hereinbelow with reference to FIGS. 8A and 8B.

Alternatively, as described hereinbelow with reference to FIGS. 9A and 9B, a body mounting body position training device of any of the types described above with reference to FIGS. 1-6B, may be mounted on a user's ankle.

As a further alternative, as described hereinbelow with reference to FIGS. 10A and 10B, a body mounting body position training device of any of the types described above with reference to FIGS. 1-6B, may be mounted on a user's knee.

As another alternative, as described hereinbelow with reference to FIGS. 11A and 11B, a body mounting body position training device of any of the types described above with reference to FIGS. 1-6B, may be mounted on a user's upper back.

As yet another alternative, as described hereinbelow with reference to FIGS. 12A and 12B, a body mounting body position training device of any of the types described above with reference to FIGS. 1-6B, may be mounted on a user's lower back.

As still another alternative, as described hereinbelow with reference to FIGS. 13A, 13B and 13C, a body mounting body position training device of any of the types described above with reference to FIGS. 1-6B, may be mounted on a user's wrist.

Alternatively, as described hereinbelow with reference to FIGS. 14A and 14B, a body mounting body position training device of any of the types described above with reference to FIGS. 1-6B, may be mounted on a user's shoulder.

As a further alternative, as described hereinbelow with reference to FIGS. 15A and 15B, a body mounting body position training device of any of the types described above with reference to FIGS. 1-6B, may be mounted on a user's neck;

As a still further alternative, as described hereinbelow with reference to FIGS. 16A and 16B, a body mounting body position training device of any of the types described above with reference to FIGS. 1-6B, may be mounted on a user's hip.

Reference is now made to FIGS. 8A and 8B, which are simplified illustrations of two body positions, which may be sensed by an elbow-mounted body position training device of FIGS. 1-6B. It is noted that body position training device 800, which may be constructed in accordance with any of the embodiments described hereinabove with reference to FIGS. 1-6B, is positioned such that two end sensors 802 and 804 are attached to the skin at locations sufficiently separated from the vertex of the elbow such that during bending of the elbow, the stretching of the skin does not displace the sensors such that they overlie the vertex.

In this example, the training instructions to the body position training device 800 preferably include at least one of:
1. Feedback generation instructions based on a predetermined operative orientation of the body-mounted body position training device 800, such as a 30 degree bend of the elbow, as shown in FIG. 8A.
2. Feedback generation instructions based on exceedance of a predetermined operative orientation threshold of the body-mounted body position training device 800, such as a bend of the elbow exceeding 90 degrees as seen in FIG. 8B.
3. Feedback generation instructions based on a reaching a plurality of predetermined operative orientations of the body-mounted body position training device 800, such as those shown in FIGS. 8A and 8B.
4. Feedback generation instructions based on a completion of a predetermined number of predetermined movements of the body-mounted body position training device 800, such as completion of each of a plurality arm bends through a predetermined angular range exemplified in FIGS. 8A and 8B.

Reference is now made to FIGS. 9A and 9B, which are simplified illustrations of two body positions which may be sensed by an ankle-mounted body position training device of FIGS. 1-6B. It is noted that body position training device 900, which may be constructed in accordance with any of the embodiments described hereinabove with reference to FIGS. 1-6B, is positioned such that two end sensors 902 and 904 are attached to the skin at locations sufficiently separated from the vertex of the ankle such that during bending of the ankle, the stretching of the skin does not displace the sensors such that they overlie the vertex.

In this example, the training instructions to the body position training device 900 preferably include at least one of:
1. Feedback generation instructions based on a predetermined operative orientation of the body-mounted body position training device 900, such as a 120 degree uplifting of the ankle, as shown in FIG. 9B.
2. Feedback generation instructions based on exceedance of a predetermined operative orientation threshold of the body-mounted body position training device 900, such as a uplifting of the ankle exceeding 90 degrees as seen in FIG. 9A.
3. Feedback generation instructions based on a reaching a plurality of predetermined operative orientations of the body-mounted body position training device 900, such as those shown in FIGS. 9A and 9B.
4. Feedback generation instructions based on a completion of a predetermined number of predetermined movements of the body-mounted body position training device 900, such as completion of each of a plurality ankle upliftings through a predetermined angular range exemplified in FIGS. 9A and 9B.

Reference is now made to FIGS. 10A and 10B, which are simplified illustrations of two body positions which may be sensed by a knee-mounted body position training device of FIGS. 1-6B. It is noted that body position training device 1000, which may be constructed in accordance with any of the embodiments described hereinabove with reference to FIGS. 1-6B, is positioned such that two end sensors 1002 and 1004 are attached to the skin at locations sufficiently separated from the vertex of the elbow such that during bending of the knee, the stretching of the skin does not displace the sensors such that they overlie the vertex.

In this example, the training instructions to the body position training device 1000 preferably include at least one of:
1. Feedback generation instructions based on a predetermined operative orientation of the body-mounted body position training device 1000, such as a 180 degree bend of the knee, as shown in FIG. 10A.
2. Feedback generation instructions based on exceedance of a predetermined operative orientation threshold of the body-mounted body position training device 1000, such as a bend of the knee exceeding 90 degrees as seen in FIG. 10B.
3. Feedback generation instructions based on a reaching a plurality of predetermined operative orientations of the body-mounted body position training device 1000, such as those shown in FIGS. 10A and 10B;
4. Feedback generation instructions based on a completion of a predetermined number of predetermined movements of the body-mounted body position training device 1000, such as completion of each of a plurality arm bends through a predetermined angular range exemplified in FIGS. 10A and 10B.

Reference is now made to FIGS. 11A and 11B, which are simplified illustrations of two body positions which may be sensed by an upper back-mounted body position training device of FIGS. 1-6B. It is noted that body position training device 1100, which may be constructed in accordance with any of the embodiments described hereinabove with reference to FIGS. 1-6B, is positioned such that two end sensors 1102 and 1104 are attached to the skin at vertebra locations sufficiently separated from each other along the spine such that during bending of the upper back, the stretching of the skin does not displace the sensors such that they overlie the vertex.

In this example, the training instructions to the body position training device 1100 preferably include at least one of:
1. Feedback generation instructions based on a predetermined operative orientation of the body-mounted body position training device 1100, such as a 30 degree bend of the upper back, as shown in FIG. 11A.
2. Feedback generation instructions based on exceedance of a predetermined operative orientation threshold of the body-mounted body position training device 1100, such as a bend of the upper back exceeding 15 degrees as seen in FIG. 11B.
3. Feedback generation instructions based on a reaching a plurality of predetermined operative orientations of the body-mounted body position training device 1100, such as those shown in FIGS. 11A and 11B.
4. Feedback generation instructions based on a completion of a predetermined number of predetermined movements of the body-mounted body position training device 1100, such as completion of each of a plurality upper back bends through a predetermined angular range exemplified in FIGS. 11A and 11B.

Reference is now made to FIGS. 12A and 12B, which are simplified illustrations of two body positions which may be sensed by a lower back-mounted body position training device of FIGS. 1-6B. It is noted that body position training device 1200, which may be constructed in accordance with any of the embodiments described hereinabove with reference to FIGS. 1-6B, is positioned such that two end sensors 1202 and 1204 are attached to the skin at vertebra locations sufficiently separated from each other along the spine, for example corresponding to L3 and L5 vertebra locations, such that during bending of the lower back, the stretching of the skin does not displace the sensors such that they overlie the vertex.

In this example, the training instructions to the body position training device 1200 preferably include at least one of:
1. Feedback generation instructions based on a predetermined operative orientation of the body-mounted body position training device 1200, such as concave bend of the lower back, as shown in FIG. 12A.
2. Feedback generation instructions based on exceedance of a predetermined operative orientation threshold of the body-mounted body position training device 1200, such as a bend of the lower back exceeding convex bend as seen in FIG. 12B.
3. Feedback generation instructions based on a reaching a plurality of predetermined operative orientations of the body-mounted body position training device 1200, such as those shown in FIGS. 12A and 12B.
4. Feedback generation instructions based on a completion of a predetermined number of predetermined movements of the body-mounted body position training device 1200, such as completion of each of a plurality arm bends through a predetermined angular range exemplified in FIGS. 12A and 12B.

Reference is now made to FIGS. 13A 13B and 13C, which are simplified illustrations of two body positions which may be sensed by an wrist-mounted body position training device of FIGS. 1-6B. It is noted that body position training device 1300, which may be constructed in accordance with any of the embodiments described hereinabove with reference to FIGS. 1-6B, is positioned such that two end sensors 1302 and 1304 are attached to the skin at locations sufficiently separated from the vertex of the wrist such that during bending of the wrist, the stretching of the skin does not displace the sensors such that they overlie the vertex.

In this example, the training instructions to the body position training device 1300 preferably include at least one of:
1. Feedback generation instructions based on a predetermined operative orientation of the body-mounted body position training device 1300, such as a 90 degree bend of the wrist, as shown in FIG. 13A or 13C.
2. Feedback generation instructions based on exceedance of a predetermined operative orientation threshold of the body-mounted body position training device 1300, such as a bend of the wrist exceeding 180 degrees as seen in FIG. 13B.
3. Feedback generation instructions based on a reaching a plurality of predetermined operative orientations of the body-mounted body position training device 1300, such as those shown in FIGS. 13A and 13C.
4. Feedback generation instructions based on a completion of a predetermined number of predetermined movements of the body-mounted body position training device 1300, such as completion of each of a plurality arm bends through a predetermined angular range exemplified in FIGS. 13A and 13C.

Reference is now made to FIGS. 14A and 14B, which are simplified illustrations of two body positions which may be sensed by a shoulder-mounted body position training device of FIGS. 1-6B. It is noted that body position training device 1400, which may be constructed in accordance with any of the embodiments described hereinabove with reference to FIGS. 1-6B, is positioned such that two end sensors 1402 and 1404 are attached to the skin at locations sufficiently separated from the vertex of the shoulder such that during bending of the shoulder, the stretching of the skin does not displace the sensors such that they overlie the vertex.

In this example, the training instructions to the body position training device 1400 preferably include at least one of:
1. Feedback generation instructions based on a predetermined operative orientation of the body-mounted body position training device 1400, the shoulder heading up of, as shown in FIG. 14B.
2. Feedback generation instructions based on exceedance of a predetermined operative orientation threshold of the body-mounted body position training device 1400, such the shoulder exceeding straight down movement as seen in FIG. 14A.
3. Feedback generation instructions based on a reaching a plurality of predetermined operative orientations of the body-mounted body position training device 1400, such as those shown in FIGS. 14A and 14C.
4. Feedback generation instructions based on a completion of a predetermined number of predetermined movements of the body-mounted body position training device 1400, such as completion of each of a plurality shoulder heading up and down, through a predetermined angular range exemplified in FIGS. 14A and 14B.

Reference is now made to FIGS. 15A and 15B, which are simplified illustrations of two body positions which may be sensed by a neck-mounted body position training device of FIGS. 1-6B. It is noted that body position training device 1500, which may be constructed in accordance with any of the embodiments described hereinabove with reference to FIGS. 1-6B, is positioned such that two end sensors 1502 and 1504 are attached to the skin at vertebra locations sufficiently separated from each other along the spine, such that during bending of the neck, the stretching of the skin does not displace the sensors such that they overlie the vertex.

In this example, the training instructions to the body position training device 1500 preferably include at least one of:
1. Feedback generation instructions based on a predetermined operative orientation of the body-mounted body position training device 1500, such as looking downwardly corresponding to concave bend of the neck, as shown in FIG. 15A.
2. Feedback generation instructions based on exceedance of a predetermined operative orientation threshold of the body-mounted body position training device 1500, such as a bend of the neck, looking upwardly, exceeding the extent of a predetermined convex bend as seen in FIG. 15B.
3. Feedback generation instructions based on a reaching a plurality of predetermined operative orientations of the body-mounted body position training device 1500, such as those shown in FIGS. 15A and 15B.
4. Feedback generation instructions based on a completion of a predetermined number of predetermined movements of the body-mounted body position training device 1500, such as completion of each of a plurality neck bends through a predetermined angular range exemplified in FIGS. 15A and 15B.

Reference is now made to FIGS. 16A and 16B, which are simplified illustrations of two body positions which may be sensed by a hip-mounted body position training device of FIGS. 1-6B. It is noted that body position training device 1600, which may be constructed in accordance with any of the embodiments described hereinabove with reference to FIGS. 1-6B, is positioned such that two end sensors 1602 and 1604 are attached to the skin at locations sufficiently separated from the vertex of the hip such that during bending of the hip, the stretching of the skin does not displace the sensors such that they overlie the vertex.

In this example, the training instructions to the body position training device 1600 preferably include at least one of:
1. Feedback generation instructions based on a predetermined operative orientation of the body-mounted body position training device 1600, such as a 90 degree bend of the hip, as shown in FIG. 16A.
2. Feedback generation instructions based on exceedance of a predetermined operative orientation threshold of the body-mounted body position training device 1600, such as a bend of the hip exceeding 120 degrees as seen in FIG. 16B.
3. Feedback generation instructions based on a reaching a plurality of predetermined operative orientations of the body-mounted body position training device 1600, such as those shown in FIGS. 16A and 16B.
4. Feedback generation instructions based on a completion of a predetermined number of predetermined movements of the body-mounted body position training device 1600, such as completion of each of a plurality arm bends through a predetermined angular range exemplified in FIGS. 16A and 16B.

It will be appreciated by persons skilled in the art that the present invention is not limited by what has been particularly claimed and includes both combinations and subcombinations of features described and shown hereinabove as well as modifications thereof which are not in the prior art.

1.-17. (canceled)

18. A method for body movement feedback comprising the steps of:
sensing at least relative positions of mutually articulated body portions of a user in real time and providing sensor outputs;
receiving said sensor outputs and providing processor outputs representing relative movements of said mutually articulated body portions; and
generating predetermined feedbacks to said user in response to predetermined movements of said mutually articulated body portions.

19. A method for body movement feedback according to claim 18 and also comprising receiving feedback selection inputs from a person and assigning selectable user feedbacks to selectable movements of said mutually articulated body portions.

20. A method for body movement feedback according to claim 18 and wherein said sensing comprises sensing the orientation of at least two body portions.

21. A method for body movement feedback according to claim 18 and wherein said sensing comprises sensing the orientation of at least one body portion and sensing bending between at least two mutually articulated body portions.

22. A method for body movement feedback according to claim 20 and wherein said sensing the orientation of at least two body portions comprises measuring mutual orientation of at least two body portions about an articulation joint joining said at least two body portions.

23. A method for body movement feedback according to claim 21 and wherein said sensing the orientation of at least one body portion and sensing bending between at least two mutually articulated body portions comprises measuring mutual orientation of at least two body portions about an articulation joint joining said at least two body portions.

24. A method for body movement feedback according to claim 18 and wherein said predetermined feedback comprises at least one of tactile feedback, auditory feedback and visual feedback.

25.-33. (canceled)
